(19) United States
(12) Patent Application Publication
(10) Pub. No.: US 2007/0255320 A1
(43) Pub. Date: Nov. 1, 2007

(54) Method and apparatus for forming insulated implantable electrodes

(75) Inventors: D. Michael Inman, Seabrook, TX (US); Steven M. Parnis, Pearland, TX (US); Albert W. Guzman, League City, TX (US)

Correspondence Address: CYBERONICS, INC., LEGAL DEPARTMENT, 6TH FLOOR, 100 CYBERONICS BOULEVARD, HOUSTON, TX, 77058, US

(73) Assignee: CYBERONICS, INC.

(21) Appl. No.: 11/414,013

(22) Filed: Apr. 28, 2006

(51) Int. Cl. A61N 1/00

(52) U.S. Cl. 607/2

(57) ABSTRACT

A method, system, and an apparatus are provided for insulating an electrode implanted in a body of a patient. The method comprises surgically exposing a nerve of the patient, implanting at least one electrode in the body of the patient, wherein the implanting comprises coupling theat least one electrode to the nerve of the patient, providing a mold form, disposing the mold form around at least a portion of the at least one electrode and the nerve, introducing into the mold form a curable liquid insulant, allowing the curable liquid insulant to cure, removing the mold form from around the at least one electrode and from the patient's body, and surgically closing the exposure of the nerve.

### BACKGROUND OF THE INVENTION

1. Field of the Invention

This invention relates generally to medical devices and, more particularly, to methods, apparatus, and systems for insulating an electrode that may selectively stimulate or sense nerve tissue activity for a medical device, such as an implantable medical device for treating or sensing a physiologic condition of a patient.

2. Description of the Related Art

Electrical and/or neural activity occurs continually throughout the human body. For example, the central nervous system (CNS) is generally a hub of electrical and/or neural activity requiring appropriate management. The human brain controls the central nervous system (CNS) in a supervisory role. Properly controlled electrical or neural activity enables the human brain to manage various mental and body functions to maintain homeostasis.

In addition to a drug regimen or surgical intervention, potential treatments for many diseases and disorders include delivery of a therapeutic signal to a target body tissue. In particular, by selectively applying therapeutic electrical signals to one or more electrodes coupled to the patient's neural tissue, a medical device such as an implantable medical device (IMD) may generate and apply an electrical signal to a target neural tissue location. In addition, such a medical device may be used to sense electrical activity in the target neural tissue to treat a patient's disease, condition or disorder.

As used herein, “stimulation” or “stimulation signal” refers to the application of an electrical, mechanical, magnetic, electromagnetic, photonic, audio and/or chemical signal to a neural structure in the patient's body. The signal is an exogenous signal that is distinct from the endogenous electrical, mechanical, and chemical activity (e.g., afferent and/or efferent electrical action potentials) generated by the patient's body and environment. In other words, the stimulation signal (whether electrical, mechanical, magnetic, electro-magnetic, photonic, audio or chemical in nature) applied to the nerve in the present invention is a signal applied from an artificial source, e.g., a neurostimulator.

A “therapeutic signal” refers to a stimulation signal delivered to a patient's body with the intent of treating a disorder by providing a modulating effect to neural tissue. The effect of a stimulation signal on neuronal activity is termed “modulation”; however, for simplicity, the terms “stimulating” and “modulating”, and variants thereof, are sometimes used interchangeably herein. In general, however, the delivery of an exogenous signal itself refers to “stimulation” of the neural structure, while the effects of that signal, if any, on the electrical activity of the neural structure are properly referred to as “modulation.” The effect of delivery of the stimulation signal to the neural tissue may be excitatory or inhibitory and may potentiate acute and/or long-term changes in neuronal activity. For example, the “modulating” effect of the stimulation signal to the neural tissue may comprise one more of the following effects: (a) changes in neural tissue to initiate an action potential (afferent and/or efferent action potentials); (b) inhibition of conduction of action potentials (whether endogenous or exogenously induced) or blocking the conduction of action potentials (hyperpolarizing or collision blocking), (c) affecting changes in neurotransmitter/neuromodulator release or uptake, and (d) changes in neuro-plasticity or neurogenesis of brain tissue.

Therapeutic electrical signals may be applied to a neural structure of the body, and more particularly to cranial nerves such as the vagus nerve. Therapeutic electrical stimulation of the vagus nerve has been used to treat epilepsy and depression. To provide a neurostimulation therapy to a patient, a neurostimulator device may be implanted in a target location in the patient's body. Such a neurostimulator device system may comprise an electrical signal generator coupled to an electrical lead(s) having one or more electrodes coupled, in turn, to a neural structure (e.g., a cranial nerve such as the vagus nerve). An electrical signal may be provided to the electrode(s) thus to the neural structure.

Electrical neurostimulation or modulation of a neural structure refers to the application of an exogenous electrical signal (as opposed to a magnetic, chemical or mechanical signal), to the neural structure. Electrical neurostimulation may be provided by implanting an electrical device underneath the skin of a patient and delivering an electrical signal to a nerve such as a cranial nerve. The electrical neurostimulation may involve performing a detection, with the electrical signal being delivered in response to a detected physiologic parameter. This type of stimulation is generally referred to as “active,” “feedback,” “closed loop,” or “triggered” stimulation. Alternatively, the system may operate without a detection system once the patient has been diagnosed with epilepsy (or another medical condition), and may periodically apply a series of electrical pulses to the nerve (e.g., a cranial nerve such as a vagus nerve) intermittently throughout the day, or over another predetermined time interval. This type of stimulation is generally referred to as “passive,” “non-feedback,” “open loop,” or “prophylactic,” stimulation. The stimulation may be applied by an implantable medical device that is implanted within the patient's body. In another alternative embodiment, the signal may be generated by an external pulse generator outside the patient's body, coupled by an RF or wireless link to an implanted electrode.

In one example of a neurostimulation system, circumneural electrodes surround a portion of a nerve longitudinally to provide electrical stimulation of the nerve. The electrical stimulation may modulate electrical signals or impulses carried by the nerve. Alternatively or additionally, an electrode may sense electrical signals carried by the nerve. “Sensing” thus refers to the detection by electrodes of the electrical activity (i.e., voltage and/or current fluctuations, whether endogenously or exogenously induced) propagating along a nerve. For example, a medical device, such as an implantable medical device may use such an electrode to stimulate or sense nerve activity on a portion of a tissue.

One problem associated with state-of-the-art neurostimulation systems is the fact that implantation of an electrode on a nerve may unintentionally affect surrounding tissue structure proximate to the nerve. Nerves and surrounding tissue structure are sensitive and may be easily damaged or traumatized by abrasion or stresses caused by implantation of an electrode. Moreover, a stimulating electrode implanted for stimulating a particular nerve may stimulate other nerves or other types of tissue structures, possibly leading to undesirable conditions. The efficiency of the treatment may also be negatively affected.

Another problem associated with state-of-the-art neurostimulation systems is that dislodging an implanted electrode from the nerve after installation nerve may cause swelling of the nerve. For example, a circumferential electrode that is attached to the nerve may inflame the surrounding tissue structure. Some physical shapes of circumneural electrodes may apply adverse mechanical forces on a targeted nerve, depriving the targeted nerve of essential nutrients. For example, cuff electrodes often cause significant nerve damage.

Yet another problem with state-of-the-art neurostimulation systems is that any undesired stimulation of the tissue structure surrounding the targeted nerve may cause unwanted effects, such as damage to the tissue. In particular, large gaps between the electrodes may allow leakage of stimulation current from an implanted electrode. The stimulation current may flow not only through a targeted nerve, but may flow outside of the nerve, and even worse, flow through tissue structures nearby in a patient's body. Such flow of leakage current through tissue structures proximate the implanted electrode may adversely affect normal functioning of non-targeted tissue regions and/or waste power in the neurostimulation system.

### SUMMARY OF THE INVENTION

In one aspect, the present invention comprises a method for insulating an electrode implanted in the body of a patient. The method comprises surgically exposing a nerve of the patient, and implanting at least one electrode in the body of the patient. The implanting step includes coupling the at least one electrode to the nerve. The method further comprises providing a mold form and disposing the form around at least a portion of the at least one electrode and the nerve. A curable liquid insulant is introduced into the mold form and allowed to cure. The mold form is removed from around the electrode and from the patient's body, and the exposure of the nerve is surgically closed.

In another aspect, the invention comprises an implanted medical device system for treating a medical condition of a patient. The system comprises a medical device implanted in the patient's body. The medical device is capable of at least one of generating a therapeutic electrical signal and sensing electrical activity on a nerve. The system further comprises an implanted electrode that is coupled to a nerve and to the implanted medical device. An in situ formed sealed insulation capsule covers the implanted electrode and at least a portion of the nerve, and acts to prevent intercellular body fluids outside the nerve from contacting the implanted electrode.

In a further aspect, the invention comprises a method of insulating an implanted electrode coupled to a nerve. The method comprises providing a lead assembly comprising at least one lead body having proximal and distal ends, and at least one electrode coupled to the distal end. The method also includes surgically exposing a cranial nerve, and implanting the lead assembly in the body of the patient. The at least one electrode is coupled to the cranial nerve. A mold form is provided and disposed around the at least one electrode and at least a portion of the cranial nerve. A liquid insulation comprising at least one of a polymer mixture and a gel is introduced into the mold, and the liquid insulation is cured to form a sealed insulation capsule around the at least one electrode. The exposure of the cranial nerve is surgically closed.

### BRIEF DESCRIPTION OF THE DRAWINGS

The invention may be understood by reference to the following description taken in conjunction with the accompanying drawings, in which like reference numerals identify like elements, and in which:

FIGS. 1A-1D are stylized diagrams of an implantable medical device implanted into a patient's body for providing stimulation to a portion of the patient's body, in accordance with one illustrative embodiment of the present invention;

FIG. 2 is a perspective view of an electrode in a mold form for providing an insulating barrier in an implantable electrode in accordance with one illustrative embodiment of the present invention.

FIG. 3 is a cross sectional view of the implantable electrode insulated within an in situ formed capsule, in accordance with one illustrative embodiment of the present invention;

FIG. 4 is a block diagram of an implantable medical device that suitable for use with the implantable electrode of FIG. 2, in accordance with one illustrative embodiment of the present invention;

FIG. 5 is a flowchart depiction of providing a long-term, in situ formed insulation for an implanted electrode coupled to a medical device, in accordance with one illustrative embodiment of the present invention;

FIGS. 5A-5B are flowcharts depicting a method of providing a long-term, in situ formed insulation for an implanted electrode coupled to a medical device, in accordance with one illustrative embodiment of the present invention; and

FIG. 6 is a flowchart depicting a method of insulating an implanted electrode coupled to a nerve using a liquid insulation which may be allowed to solidify or thicken to provide a sealed insulation capsule in accordance with one illustrative embodiment of the present invention.

While the invention is susceptible of various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims.

### DETAILED DESCRIPTION OF SPECIFIC EMBODIMENTS

Illustrative embodiments of the invention are described herein. In the interest of clarity, not all features of an actual implementation are described in this specification. In the development of any such actual embodiment, numerous implementation-specific decisions must be made to achieve the design-specific goals, which will vary from one implementation to another. It will be appreciated that such a development effort, while possibly complex and time-consuming, would nevertheless be a routine undertaking for persons of ordinary skill in the art having the benefit of this disclosure.

Generally, the present invention relates to providing a long-term, in situ formed insulation for an implanted electrode coupled to a medical device. A method comprises providing at least one lead having a proximal end and at least one electrode at a distal end thereof. A neural structure is surgically exposed, and the at least one electrode is coupled to the neural structure. A medical device capable of providing an electrical signal is provided, and the proximal end of the at least one lead is coupled to the medical device. An insulation form is provided that surrounds at least a portion of the at least one electrode and at least a portion of the neural structure. A liquid insulation comprising at least one of a polymer mixture and a gel is delivered into the insulation form and allowed to solidify and/or thicken to provide an in situ formed insulation. The invention further comprises surgically closing the exposure of the neural structure and allowing the electrode to remain in the patient's body for a predetermined time period.

In another aspect, the invention comprises a method of insulating an implanted electrode coupled to a nerve. The method comprises providing a lead assembly comprising at least one lead body having proximal and distal ends, and at least one electrode coupled to the distal end. A neural structure, such as a nerve including a cranial nerve is surgically exposed and the at least one electrode is coupled to the nerve. A liquid insulation comprising at least one of a polymer mixture and a gel is applied to cover the at least one electrode and at least a portion of the nerve. The liquid insulation is allowed to solidify or thicken to provide a sealed insulation capsule preventing intercellular body fluids outside the neural structure from contacting the at least one electrode. The invention further comprises surgically closing the exposure of the nerve and allowing the electrode to remain in the patient's body for a predetermined time period.

In another aspect, the invention comprises a method of insulating an implanted electrode coupled directly to a stimulator (e.g., an electrical pulse generator) that does not have a lead body. A neural structure, e.g., a nerve such as a cranial nerve, is surgically exposed and the at least one electrode of the leadless stimulator is coupled to the neural structure. A liquid insulation comprising at least one of a polymer mixture and a gel is applied to cover the at least one electrode of the leadless stimulator and at least a portion of the neural structure. The liquid insulation is allowed to solidify or thicken to provide a sealed insulation capsule preventing intercellular body fluids outside the neural structure from contacting the at least one electrode. The invention further comprises surgically closing the exposure of the nerve and allowing the electrode to remain in the patient's body for a predetermined time period.

An implantable medical device system is provided for treating a patient with a medical condition. An implantable medical device may generate a therapeutic electrical signal and/or sense electrical activity on a nerve. An electrode attached to a nerve may be operatively coupled to the implantable medical device. An in situ formed sealed insulation capsule covers the electrode and at least a portion of the nerve to prevent intercellular body fluids outside the neural structure from contacting the electrode.

Examples of electrodes include an insulated electrode, such as a circumferential neural (circumneural) electrode for implantation on a selected neural tissue of a patient. The insulated electrode may be adapted to selectively provide a therapeutic electrical signal and/or to detect electrical signals on a nerve. Some embodiments of the present invention use the insulated electrode for applying an electrical signal from an implantable medical device (IMD) to a nerve of a patient. A therapeutic electrical signal applied to the nerve using the insulated electrode may provide a desired therapeutic effect without affecting unintended portions of the nerve fibers or generating additional unintentional (exogenously induced) electrical activity on the nerve, or both.

The implantable medical device may be an implantable medical device that is capable of providing an electrical signal to the insulated electrode for modulating the electrical activity on the nerve to provide a therapeutic effect. Some embodiments of the present invention provide for methods, apparatus, and systems to selectively provide an electrical signal to a nerve of a patient using a multi-channel electrode. Other embodiments of the present invention provide for methods, apparatus, and systems to selectively sense (i.e., detect) an electrical signal on a nerve of a patient using a multi-channel electrode. In certain embodiments, the nerve comprises a cranial nerve, and more preferably a vagus nerve. In this way, the insulated electrode may selectively provide stimulation to a nerve, such as the vagus nerve (cranial nerve X), from an implantable medical device (IMD), such as a neurostimulator, to treat a disorder or a medical condition.

An implantable medical device system for treating a patient with a medical condition may comprise an implantable medical device for generating an electrical signal. The implantable medical device system further comprises an electrode operatively coupled to the implantable medical device for delivering the electrical signal to a portion of a tissue. The same or a different electrode may be used to sense electrical activity on a tissue. Embodiments of the present invention provide for an insulated electrode which may be used with a neurostimulator system for treatment of disorders such as neuropsychiatric disorders (e.g., depression), movement disorders (e.g., epilepsy, Parkinson's disease), eating and/or gastric-related disorders (e.g., obesity/compulsive overeating, bulimia nervosa, anorexia nervosa), a endocrine disorders (e.g., diabetes), reproductive disorders (e.g., infertility), metabolic disorders, hearing disorders, chronic pain, and/or a cardiac disorders, among others.

The implantable medical device may comprise a controller to selectively provide a stimulation signal to the electrode, or detect a signal using the electrode. The term “electrode” may refer to a single electrode, or may refer to a plurality of insulated electrodes, each insulated electrode corresponding to a stimulation or sensing channel. The controller may be capable of selecting one or more channels for the plurality of insulated electrodes.

Persons of skill in the art will appreciate that many electrode designs could be used in the present invention. Structurally, an electrode may comprise at least one insulated electrode comprising a conductive material such as platinum, iridium, platinum-iridium alloys, titanium, and/or oxides of the foregoing. Examples of the insulated electrodes include electrode ribbons, spiral electrodes, wire electrodes, and helical electrodes. The case, or shell, of an electrical signal generator may also be used as an electrode. The electrode may sense or detect any target parameter in the patient's body. For example, the electrode coupled to the patient's vagus nerve may detect an intrinsic neural signal. The electrode may sense or detect an electrical signal (e.g., a voltage indicative of intrinsic neural electrical activity). The electrode, in some embodiments of the present invention, may administer a therapeutic electrical signal to the vagus nerve. Some embodiments of the present invention utilize a continuous, periodic or intermittent stimulation signal applied to the vagus nerve.

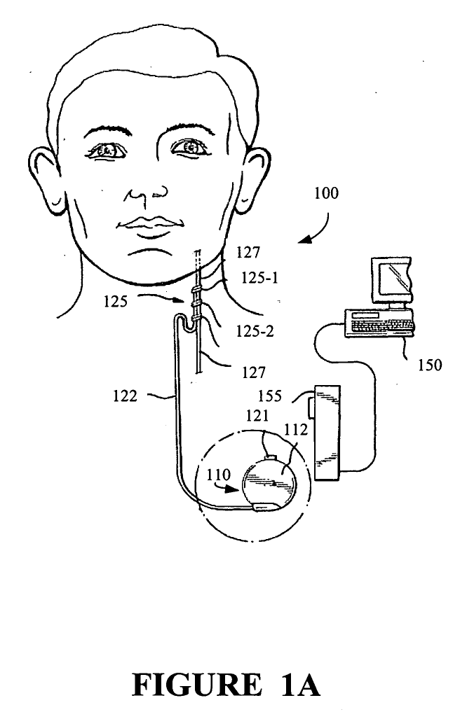

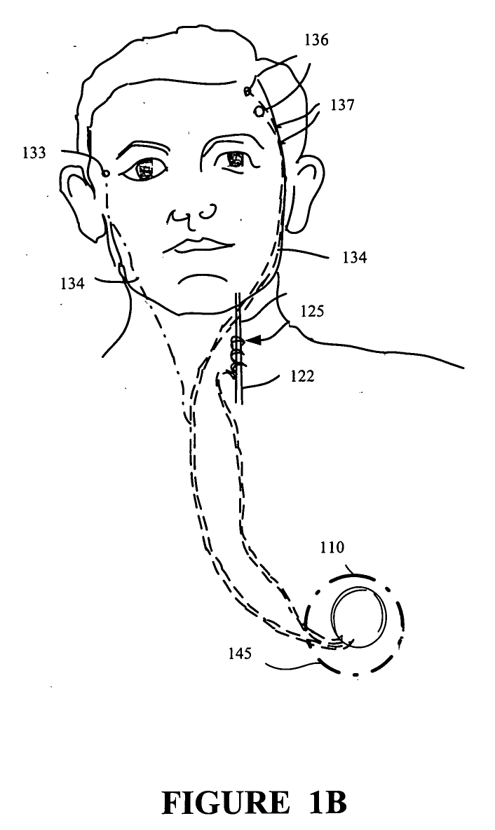

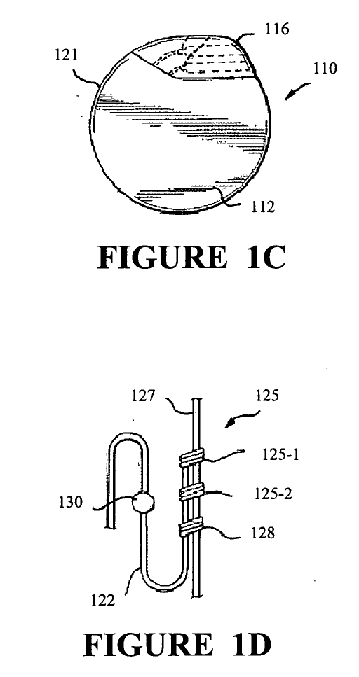

An exemplary IMD that may be implanted into a patient's body for providing a signal to a portion of the patient's body is described below according to one illustrative embodiment of the present invention. FIGS. 1A-1D depict a stylized implantable medical system 100 for implementing one or more embodiments of the present invention. FIGS. 1A-1D illustrate an electrical signal generator 110 having a main body 112 comprising a case or shell 121 (FIG. 1A) with a header 116 (FIG. 1C) for connecting to leads 122. The electrical signal generator 110 is implanted in the patient's chest in a pocket or cavity formed by the implanting surgeon just below the skin (indicated by a dotted line 145, FIG. 1B), similar to the implantation procedure for a pacemaker pulse generator.

A stimulating nerve electrode assembly 125, preferably comprising an electrode pair, is conductively coupled to the distal end of an insulated, electrically conductive lead assembly 122, which preferably comprises a pair of lead wires (one wire for each electrode of an electrode pair). Lead assembly 122 is conductively coupled at its proximal end to the connectors on the header 116 (FIG. 1C) on case 121. The electrode assembly 125 may be surgically coupled to a vagus nerve 127 in the patient's neck or at another location, e.g., near the patient's diaphragm. The electrical signal may alternatively be applied to other cranial nerves. The electrode assembly 125 preferably comprises a bipolar stimulating electrode pair 125-1, 125-2 (FIG. 1D), such as the electrode pair described in U.S. Pat. No. 4,573,481 issued Mar. 4, 1986 to Bullara. Suitable electrode assemblies are available from Cyberonics, Inc., Houston, Tex. as the Model 302 electrode assembly. However, persons of skill in the art will appreciate that many electrode designs could be used in the present invention including, e.g., paddle electrodes. Referring again to FIG. 1D, the two electrodes are preferably wrapped about the vagus nerve 127, and the electrode assembly 125 may be secured to the nerve 127 by a spiral anchoring tether 128 (FIG. 1D) such as that disclosed in U.S. Pat. No. 4,979,511 issued Dec. 25, 1990 to Reese S. Terry, Jr. and assigned to the same assignee as the instant application. Lead assembly 122 is secured, while retaining the ability to flex with movement of the chest and neck, by a suture connection 130 to nearby tissue.

In one embodiment, the open helical design of the electrode assembly 125 (described in detail in the above-cited Bullara patent), which is self-sizing and flexible, minimizes mechanical trauma to the nerve and allows body fluid interchange with the nerve. The electrode assembly 125 preferably conforms to the shape of the nerve, providing a low stimulation threshold by allowing a large stimulation contact area with the nerve. Structurally, the electrode assembly 125 comprises two electrode ribbons (not shown), of a conductive material such as platinum, iridium, platinum-iridium alloys, and/or oxides of the foregoing. The electrode ribbons are individually bonded to an inside surface of an elastomeric body portion of the two spiral electrodes 125-1 and 125-2 (FIG. 1D), which may comprise two spiral loops of a three-loop helical assembly. The lead assembly 122 may comprise two distinct lead wires or a coaxial cable whose two conductive elements are respectively coupled to one of the conductive electrode ribbons. One suitable method of coupling the lead wires or cable to the electrodes 125-1 and 125-2 comprises a spacer assembly such as that disclosed in U.S. Pat. No. 5,531,778, although other known coupling techniques may be used.

The elastomeric body portion of each loop is preferably composed of silicone rubber, and the third loop 128 (which typically has no electrode) acts as the anchoring tether 128 for the electrode assembly 125.

In certain embodiments of the invention, sensors such as eye movement sensing electrodes 133 (FIG. 1B) may be implanted at or near an outer periphery of each eye socket in a suitable location to sense muscle movement or actual eye movement. The electrodes 133 may be electrically connected to leads 134 implanted via a catheter or other suitable means (not shown) and extending along the jaw line through the neck and chest tissue to the header 116 of the electrical signal generator 110. When included in systems of the present invention, the sensing electrodes 133 may be utilized for detecting rapid eye movement (REM) in a pattern indicative of a disorder to be treated, as described in greater detail below. The detected indication of the disorder can be used to trigger active stimulation.

Other sensor arrangements may alternatively or additionally be employed to trigger active stimulation. Referring again to FIG. 1B, EEG sensing electrodes 136 may optionally be implanted and placed in spaced-apart relation on the skull, and connected to leads 137 implanted and extending along the scalp and temple, and then connected to the electrical signal generator 110 along the same path and in the same manner as described above for the eye movement electrode leads 134. In alternative embodiments, temperature-sensing elements and/or heart rate sensor elements may be employed to trigger active stimulation.

In contrast to active stimulation embodiments, other embodiments of the present invention utilize passive stimulation to deliver a continuous, periodic or intermittent electrical signal to the vagus nerve according to a programmed on/off duty cycle without the use of sensors to trigger therapy delivery. Both passive and active stimulation may be combined or delivered by a single IMD according to the present invention. Either or both modes may be appropriate to treat the particular disorder diagnosed in the case of a specific patient under observation.

The electrical signal generator 110 may be programmed with an external computer 150 using programming software of the type copyrighted by the assignee of the instant application with the Register of Copyrights, Library of Congress, or other suitable software based on the description herein, and a programming wand 155 to facilitate radio frequency (RF) communication between the computer 150 (FIG. 1A) and the pulse generator 110. The wand 155 and software permit non-invasive communication with the generator 110 after the latter is implanted. The wand 155 is preferably powered by internal batteries, and provided with a “power on” light to indicate sufficient power for communication. Another indicator light may be provided to show that data transmission is occurring between the wand and the generator.

By providing the stimulation therapy, the electrical signal generator 110 may treat a disorder or a medical condition. A generally suitable form of neurostimulator for use in the method and apparatus of the present invention is disclosed, for example, in U.S. Pat. No. 5,154,172, assigned to the same assignee as the present application. A commercially available example of such a neurostimulator is the NeuroCybernetic Prosthesis (NCP®, Cyberonics, Inc., Houston, Tex., the assignee of the present application). Certain parameters of the electrical signal generated by the electrical signal generator 110 are programmable, such as by means of an external programmer in a manner conventional for implantable electrical medical devices.

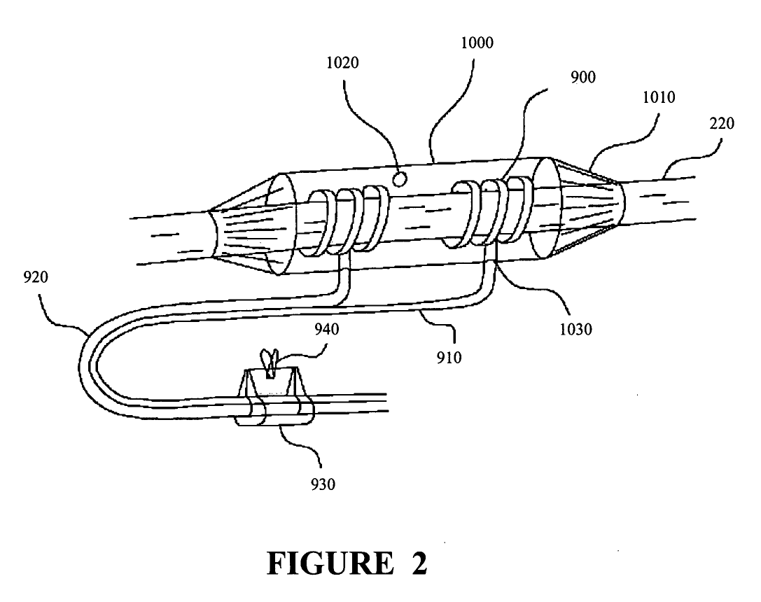

Turning to FIG. 2, a perspective view of an injectable insulation electrode in a circumneural mold form is depicted for forming an insulating barrier in an implantable electrode in accordance with one illustrative embodiment of the present invention. The circumneural mold form may be disposed circumferentially around conventional helical electrodes 900, which may be wrapped around the nerve 220 and connected to a flexible lead wire 910. The lead wire 910 may be formed into a loop or bend, such as a bend 920 of about 180 degrees, and secured to fascia or other desired structures in the body with a securing member according to known techniques, such as a flexible tie down 930 and suture 940.

The circumneural mold form may comprise an appropriately sized cylindrical shell 1000, which may be rigid, semi-rigid or flexible and made of biocompatible materials known in the art. The mold form may be fitted with two end cuffs, such as tapered and flexible cuffs 1010, to fit around the electrode site and allow the nerve 220 and lead wire 910 to pass through openings 1030, which may be close-fitting openings. For example, a desired thickness of this insulation may be one having a certain minimum thickness to assure integrity and longevity while remaining as thin as possible to retain flexibility. The shell 1000 provides a form for creating an insulation capsule around an electrode engaging a nerve, and to hold the electrode in place while the capsule is created in situ. The thickness and shape or contour of the insulation capsule may be based on variations in the size of the vagus nerve or other target nerve upon which the shell 1000 may be used to create an insulation capsule.

An injection port 1020 may be provided in shell 1000 to receive liquid epoxy/adhesive or other injectable insulant. The insulant may be allowed to cure until firm and the mold (1000/1010) is removed. Curing of the insulant may comprise a chemical reaction such as a cross-linking reaction between two polymers, evaporation of a solvent, changes in viscosity, combinations of the foregoing, and/or other chemical or physical changes, depending upon a particular application. A mold release compound may be used to aid in this step of the process.

Consistent with one embodiment, the resulting insulant form may be used to provide a capsule of insulated material that is essentially cylindrical and sufficiently large to insulate the electrodes while avoiding excessive size or pointed protrusions or structural anomalies that could cause pain or tissue damage. In this manner, the form may be used to fashion an insulating capsule for the electrodes that is comfortable for the patient. In-situ forming of an insulation layer may ensure a properly sized fit to the nerve 220, without applying excessive (and potentially uncomfortable or injurious) clamping force to the electrodes 900 and nerve 200. A substantially complete insulation around the electrodes 900 may increase the ability to sense endogenous and evoked compound action potentials in the nerve 220, in part by reducing ingress of intercellular body fluids from outside the nerve to the electrodes 900. Additionally, substantially complete insulation around the electrode sites may reduce current spread or leakage to surrounding tissues during stimulation of the target tissues. A secure attachment of the electrodes 900 to the nerve 220 may reduce or eliminate the need for a third “anchor” helical. In this manner, a combination with the flexible lead wire 910, strain relief bend 920 and/or fascia anchor may substantially reduce stress/strain on the insulant material, which may increase the lifespan of the insulation layer.

By using an insulant material, in one embodiment, a circumneural molded capsule of insulation may be provided in-situ to insulate sensing and/or stimulation electrodes 900. The circumneural capsule may be formed by a process of injecting an insulant in a cylindrical form surrounding one or more electrode(s), such as conventional helical electrodes. Conventional helical electrodes may be modified to be more compatible with the in-situ molding process.

According to one embodiment, an injectable insulant material may be used to seal in-situ sensing electrodes and/or stimulation electrodes 900. For example, by injecting the insulant material in a cylindrical shell mold all around the sensing and/or stimulation electrodes 900, a sealed structure having a controlled shape and size may be formed to substantially embed the sensing and/or stimulation electrodes 900. Alternatively, the insulant material may be applied to the back side of the electrodes to form a capsule that conforms to the shape of the surrounding tissue and voids. By forming the lead wire 120 having an approximately 180 degree bend and securing it to a fascia anchor or other desired structures in the body with the flexible tie down 1030 and the suture 1040, the electrodes 900 in combination with the in-situ formed insulator may reduce the possibility of damage to the insulator.

In one embodiment, the insulant material is allowed to cure until it is substantially firm before removing the shell mold 1000. A sealed capsule having a controlled outer surface shape may thus be provided around a living nerve bundle. For the electrodes 900, the insulant may be used as a mechanical anchor to help attach the electrodes 900 to the nerve 220, instead of or in addition to the use of a third “anchor” helical. By providing a combination of the flexible lead wire 110, the strain relief bend 1020 and a fascia anchor (based on 1040 and 1030), the insulant may reduce the stress/strain on the electrodes 900.

In-situ forming the insulant material layer to seal the sensing and/or stimulation electrodes 900 to a size that fits to the subject nerve 220 may aid in this process. That is, the portion of the insulant not facing the nerve 220 may provide control of the thickness and outer shape of the insulant capsule. The circumneural mold form may use flexible cuffs 1010 to taper the thicker portion down to the approximate size of the nerve 220.

In another embodiment, the lead wires may run along the nerve 220 itself, as with some conventional electrodes, and exit at the flexible cuff 110 alongside the nerve 220. This may provide the advantage of making the cuff simpler to construct and easier to use. The shell 1000 as a temporary structure may be placed around the nerve 220 to hold the liquid in place while it solidifies or thickens. In one embodiment of the present invention, the shell 1000 comprises a biocompatible material that may be left in the body. However, after the liquid is cured the shell 1000 may be removed in some embodiments, leaving only one type of material implanted in the body. The shell 1000 enables positioning the electrodes 900 in place. By filling the shell 1000 with the insulant material, allowing the material to thicken and then taking the shell away, the shell may provide a desired form. Alternately, the shell itself may be permanently implantable.

By routing the electrodes 900 along the surface of the nerve 220, fewer holes may be needed in shell 1000 since a hole in the exterior circumference of the shell would not be needed. That is, the lead associated with a particular electrode may exit in a manner that enables routing through the cuff 1010 instead of the exterior periphery of shell 1000. Then the lead wire may come out adjacent the nerve 220.

Yet another embodiment may combine with the leads leaving the helical electrode tangentially and taking a gentle bend away from the nerve 220 while still running tangentially, then exiting the insulant form at a thicker portion, possibly with extra reinforcing material at the point the lead exits the capsule, thereby providing additional strain relief on the exit point.

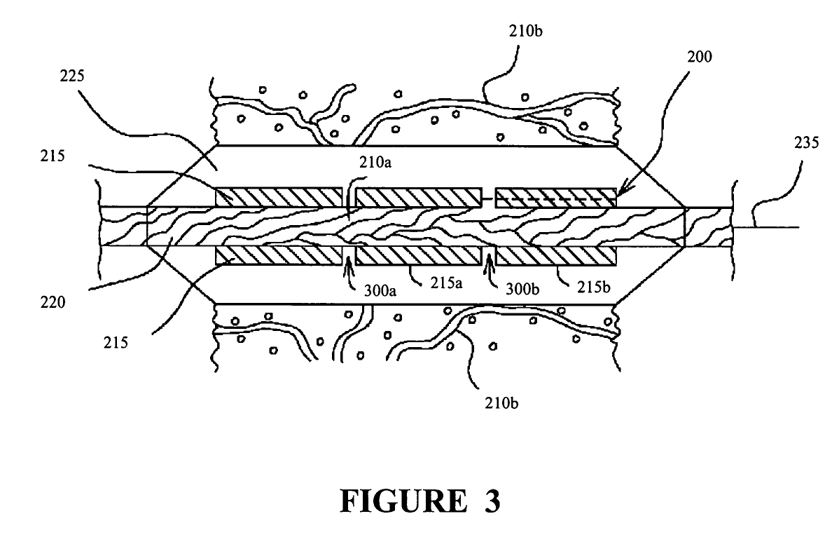

Turning now to FIG. 3, a stylized cross sectional view of an implantable electrode 200 is depicted in accordance with one illustrative embodiment of the present invention. The electrode 200 may comprise a plurality of insulated electrodes including a circumferential insulated electrode 215, and insulated electrodes 215a, and 215b in which at least two insulated electrodes may selectively receive a neurostimulating signal applied to the electrode 200. The electrode 200 may be a circumneural electrode such that an insulator 225 may substantially surround a first tissue 210a of a nerve 220 proximal to a second tissue 210b in the patient's body.

In one embodiment, at least two implantable electrodes, such as the insulated electrodes 215, 215b may be implanted in a living body for selectively driving the electrode 200 as a unipolar or a bipolar electrode. For example, in a bipolar stimulating electrode configuration, the insulated electrode 215 may be driven as one of the bipolar stimulating electrodes, while the insulated electrode 215b is used as the other bipolar stimulating electrode. Alternatively, the insulated electrode 215 may be driven as one of the bipolar stimulating electrodes, while the insulated electrode 215a is used as the other bipolar stimulating electrode; in this way different nerve fibers may be stimulated.

The electrode 200, such as a circumneural electrode, may selectively deliver an electrical signal to a patient's body and/or or detect a signal in the patient's body in accordance with one or more illustrative embodiments of the present invention. Consistent with one embodiment, the electrode 200 may comprise at least two insulated electrodes, such as a pair of cylindrical electrodes that may be spaced apart. Examples of the tissue include any nerve fibers, such as a nerve 220.

The electrode 200 may be implanted within the patient's body to provide neurostimulation or neuromodulation to the nerve 220. For example, the electrode 200 may neuromodulate the electrical activity of the vagus nerve. Alternatively, or selectively, the electrode 200 may sense a neuropotential associated with the nerve 220.

In a particular embodiment, a pain therapy may be administered by applying an electrical signal to the patient's vagus nerve by an application of an electrical stimulation signal to the nerve 220 to excite primarily the small afferent nerve fibers. In this way, the electrode 200 may enable the pain therapy to relieve neuropathic, psychogenic pain, and/or nociceptive pain where the patient is suffering from a terminal disease. To this end, the electrode 200 may enable vagus nerve stimulation (VNS) therapy in the patient's neck, i.e., the cervical region. The electrode 200 may deliver an electrical signal to the selected neural structure, such as a nerve including a cranial nerve, manually or automatically. The electrode 200 may deliver the signal continuously, periodically or intermittently when activated.

For example, neurostimulation may be delivered as a pulsed electrical signal in discrete stimulation periods known as pulse bursts, which constitute a series of controlled electrical pulses defined by a plurality of parameters. The neurostimulation signal may be generated by an electrical pulse generator and applied to the nerve 220 via the electrode 200. The parameters defining the neurostimulation signal may include a current magnitude, a pulse width, a pulse frequency, an on-time and an off-time.

However, in some embodiments, to provide vagus nerve stimulation (VNS) therapy, a patient's medical condition may also be monitored. Sensing-type electrodes, such as the electrodes 200 may be implanted at or near the vagus nerve. Using the sensing electrodes(s) 200, the patient's medical condition may be detected and associated data may be measured against a predetermined threshold level. If the patient's medical condition exceeds the predetermined threshold level over a given period, a therapeutic electrical neurostimulation signal may be applied. The therapeutic electrical neurostimulation signal may be applied periodically or applied as a result of patient intervention by manual activation using external control.

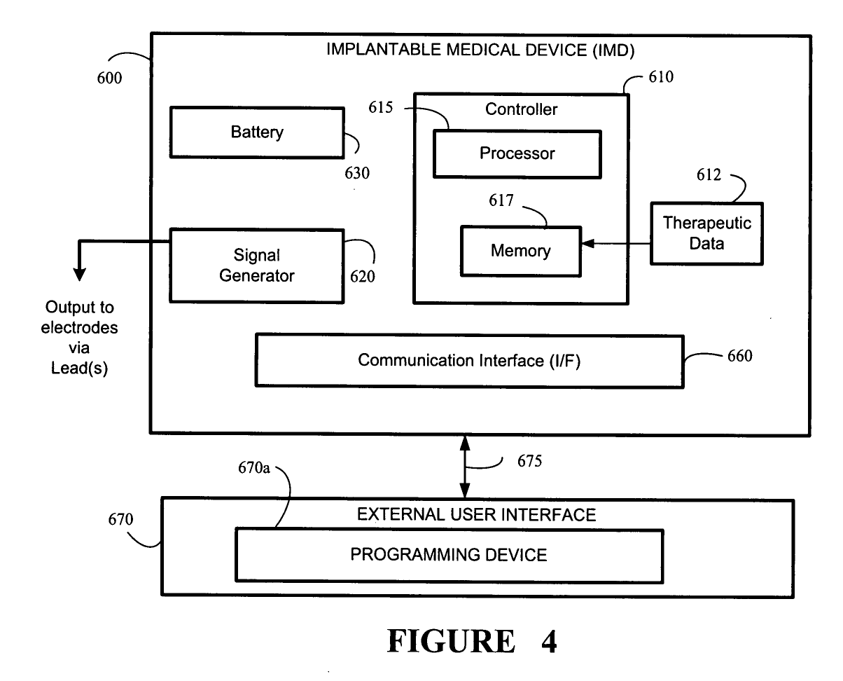

Turning now to FIG. 4, a block diagram is provided depicting an implantable medical device (IMD) 600 and an external user interface (I/F) 670, in accordance with one illustrative embodiment of the present invention. The IMD 600 may include the implantable electrode 200 of FIG. 2A. An in situ formed sealed insulation capsule may cover the electrode 200 and at least a portion of the nerve 220 to prevent intercellular body fluids outside the neural structure from contacting the electrode 200 in accordance with one illustrative embodiment of the present invention. The IMD 600 may be used to provide electrical stimulation to body tissue, such as nerve tissue, to treat various disorders, such as epilepsy, depression, bulimia, etc. The IMD 600 may be used to treat neuromuscular, neuropsychiatric, cognitive, autonomic, sensory disorders, and other medical conditions.

The IMD 600 may be coupled to various leads, such as lead assembly 122, shown in FIG. 1A. Electrical signals from the IMD 600 may be transmitted via the leads 122 to stimulation electrodes associated with the electrode assembly 125. In addition, where sensors are employed, signals from sensor electrodes may travel by leads, such as leads 122, 134 and/or 137, to the IMD 600.

The IMD 600 may comprise a controller 610 that is capable of controlling various aspects of the operation of the IMD 600. The controller 610 is capable of receiving therapeutic data 612 including internal data and/or external data to deliver the therapeutic electrical signal to at least one target portion of the human body. For example, the controller 610 may receive manual instructions from an operator externally, or it may perform stimulation based on internal calculations and protocols programmed into or resident in the IMD 600. The controller 610 is preferably capable of affecting substantially all functions of the IMD 600.

The controller 610 may comprise various components, such as a processor 615, a memory 617, and other structures conventional known to those skilled in the art having benefit of the present disclosure. The processor 615 may comprise one or more microcontrollers, microprocessors, etc., that are capable of performing various executions of software components. The memory 617 may comprise various memory portions where the therapeutic data 612 and a number of types of data (e.g., internal data, external data instructions, software codes, status data, diagnostic data, etc.) may be stored and retrieved. The memory 617 may comprise random access memory (RAM), dynamic random access memory (DRAM), electrically erasable programmable read-only memory (EEPROM), flash memory, etc. In one embodiment, the memory 617 may comprise RAM and Flash memory components.

The IMD 600 may also comprise an electrical signal generator 620. The signal generator 620 is capable of generating and delivering a variety of electrical neurostimulation signals to one or more electrodes via leads. A number of lead assemblies 122 may be coupled to the IMD 600. Therapy may be delivered to the lead(s) by the electrical signal generator 620 based upon instructions from the controller 610. The electrical signal generator 620 may comprise various circuitry, such as stimulation signal generators, and other circuitry that receives instructions relating to the type of stimulation to be performed. The electrical signal generator 620 is capable of delivering a controlled current neurostimulation signal over the leads. In one embodiment, the controlled current neurostimulation signal may refer to a prescribed or pre-determined current to a neural tissue of a patient.

The IMD 600 may also comprise a battery 630. The battery 630 may comprise one or more cells, voltage regulators, etc., to provide power for the operation of the IMD 600, including delivering stimulation. The battery 630 may comprise a power supply source that in some embodiments is rechargeable. The battery 630 provides power for the operation of the IMD 600, including electronic operations and the stimulation function. The battery 630, in one embodiment, may comprise a lithium/thionyl chloride cell or, more preferably, a lithium/carbon monofluoride (LiCFx) cell. It will be apparent to persons of skill in the art that other types of power supplies, e.g., high charge-density capacitors, may also be used instead of (or in addition to) the battery 630.

The IMD 600 also comprises a communication interface (I/F) 660 capable of facilitating communications between the IMD 600 and various devices. The communication interface 660 is capable of providing transmission and reception of electronic signals to and from the external user interface 670. The external user interface 670 may be a handheld device, preferably a handheld computer or PDA, but may alternatively comprise any other device that is capable of electronic communications and programming.

The external user interface 670 may comprise a programming device 670a that is capable of programming various modules and stimulation parameters of the IMD 600. In one embodiment, the programming device 670a is capable of executing a data-acquisition program. The programming device 670a may be controlled by a medical professional, such as a physician, at a base station in, for example, a doctor's office. The programming device 670a may download various parameters and program software into the IMD 600 for programming and controlling its operation. The programming device 670a may also receive and upload various status conditions and other data from the IMD 600.

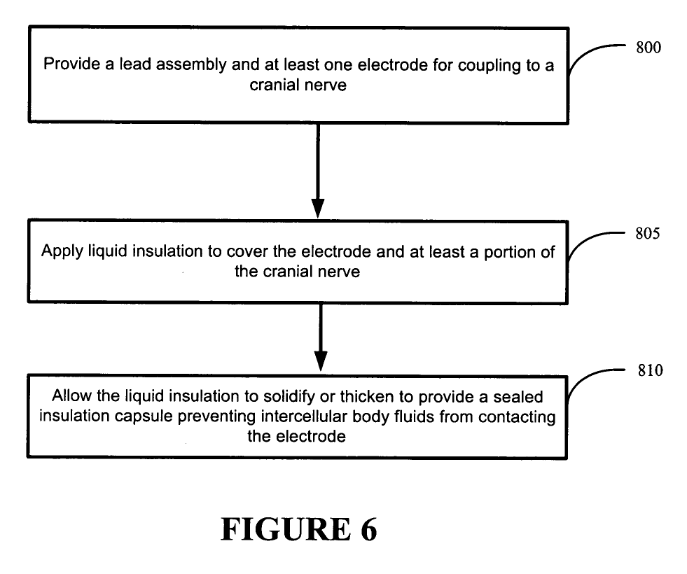

The communication user interface 660 may comprise hardware, software, firmware, and/or any combination thereof. Communications between the external user interface 670 and the communication user interface 660 may occur via a non-invasive, wireless or other type of communication, illustrated generally by line 675 in FIG. 6. Various software and/or firmware applications may be loaded into the programming device 670a for programming the external user interface 670 for communications with the IMD 600. In one embodiment, the external user interface 670 may be controlled by an operating system suitable for use in a handheld device, such as Windows® Pocket PC 2002 or Windows® Mobile 2005 operating systems offered by Microsoft Corporation of Redmond, Wash.

The IMD 600 may deliver a neurostimulation signal to the nerve 220 or a nerve fascicle within the nerve trunk. In one embodiment of the present invention, methods, apparatus, and systems provide the neurostimulation signal in a neural structure, such as a cranial nerve, and more preferably a vagus nerve. By using the neurostimulation signal, the IMD 600 may provide a neurostimulation therapy to a patient, according to one embodiment of the present invention. In one embodiment, such stimulating or modulating signals are applied to the nerve 220 via the electrode 200, and intrinsic nerve signals may be detected by the electrode 200 for processing in sense circuitry, by a signal generator.

Consistent with one embodiment, the IMD 600 may be a neurostimulator device capable of treating a disease, disorder or condition by providing electrical neurostimulation therapy to a patient. To this end, the implantable medical device 600 may be implanted in the patient at a suitable location to treat a depression disorder, an epilepsy disorder, a gastric-related disorder, a hormonal disorder, a reproductive disorder, a metabolic disorder, a hearing disorder, a pain disorder, and/or a heart rhythm disorder.

The IMD 600 may treat or control medical, psychiatric or neurological disorders in a patient. The IMD 600 may provide treatment of neurological or neurologically related diseases or disorders. To this end, the IMD 600 may provide stimulation for at least one of the trigeminal, glossopharyngeal, and vagus nerves, or other parasympathetic and/or sympathetic nerves, may improve the in patients suffering from different neurological or neurologically related diseases or disorders.

Implantable medical devices 600 that may be used in the present invention include any of a variety of electrical stimulation devices, such as a neurostimulator capable of stimulating a neural structure in a patient, especially for stimulating a patient's cranial nerve such as a vagus nerve. Although the IMD 600 is described in terms of cranial nerve stimulation, and particularly vagus nerve stimulation (VNS), a person of ordinary skill in the art would recognize that the present invention is not so limited. For example, the IMD 600 may be applied to the stimulation of other cranial nerves, such as the trigeminal and/or glossopharyngeal nerves, or other neural tissue, such as one or more brain structures of the patient, spinal nerves, and other spinal structures. In one alternative embodiment, the invention may be implemented in a spinal cord stimulator (SCS).

The IMD 600 may be a single device or a pair of devices, is implanted and electrically coupled to the lead(s) 235, which are in turn coupled to the electrode(s) 200 implanted on the left and/or right branches of the vagus nerve, for example. In one embodiment, the electrode 200 may include a set of stimulating electrode(s) separate from a set of sensing electrode(s). In another embodiment, the same electrode may be deployed to stimulate and to sense. A particular type or a combination of electrodes may be selected as desired for a given application. For example, an electrode suitable for coupling to a vagus nerve may be used. The electrodes 200 preferably comprise a bipolar stimulating electrode pair.

Using the electrode 200, the IMD 600 may apply a predetermined sequence of electrical pulses to the selected neural structure, such as a nerve including a cranial nerve to provide therapeutic neurostimulation for the patient with a disease or a disorder. While the selected cranial nerve may be the vagus nerve, the electrode 200 may comprise at least one nerve electrode for implantation on the patient's vagus nerve for direct stimulation thereof.

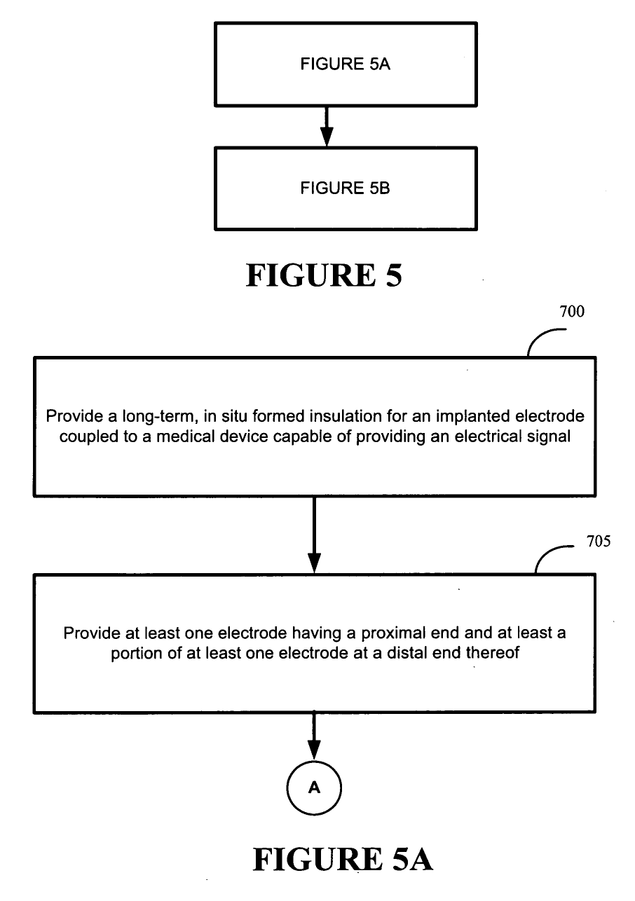

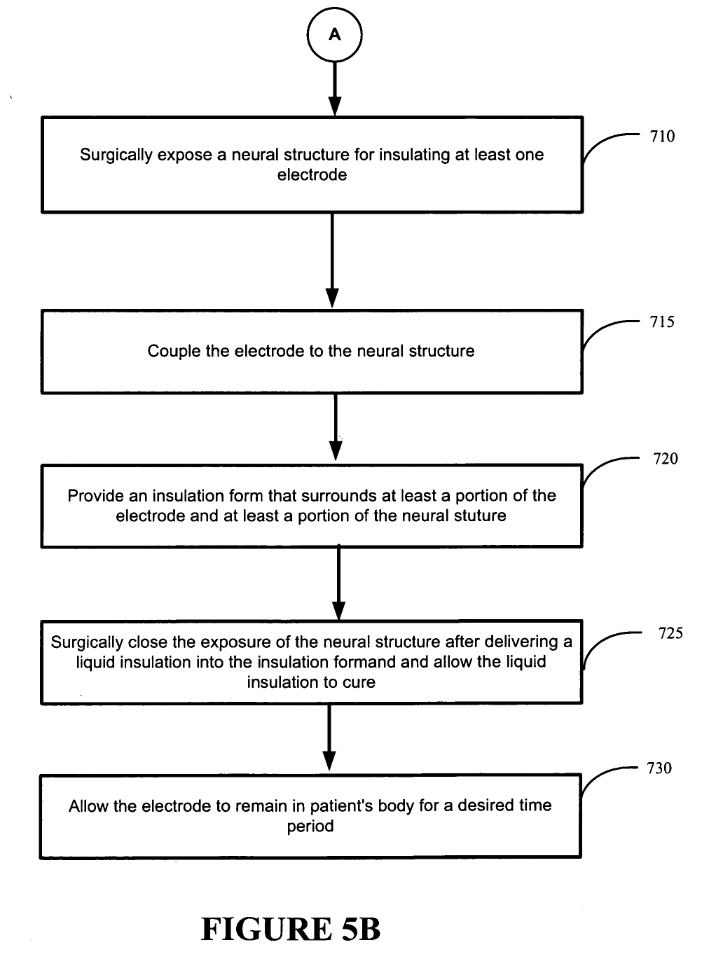

Referring to FIGS. 5, 5A and 5B, a stylized representation for implementing a method of insulating an implanted electrode is illustrated, according to one embodiment of the present invention. At Block 700, in situ formed insulation for a long term use may be provided for an implanted electrode, such as the electrode 200 shown in FIG. 2. The implanted electrode may be coupled to a medical device, such as the IMD 600 shown in FIG. 4. The medical device, or the IMD 600, may be capable of providing an electrical signal to nerve 220, as shown in FIG. 3.

At Block 705, at least one lead having a proximal end may be provided, such as the lead assembly 235 shown in FIG. 2. The lead may couple to at least a portion of an implanted electrode at a distal end thereof. The implanted electrode may be coupled to a neural structure, such as the nerve 220. The proximal end of the lead may be coupled to a medical device, such as the IMD 600.

Referring to FIG. 5B, a stylized representation for implementing a method of insulating an implantable electrode that couples to a portion of a tissue for medical device, such as the IMD 600, is illustrated, in accordance with one embodiment of the present invention. At Block 710, a neural structure, such as the nerve 220, may be surgically exposed for insulating at least one electrode, such as the electrode 200 shown in FIG. 3. At Block 715, the electrode 200 may be coupled to the neural structure. An insulation form may be provided at Block 720. The insulation form may surround at least a portion of the electrode 200 an at least a portion of the neural structure, i.e., the nerve 220.

After delivering a liquid insulation into the insulation form and allowing the liquid insulation to cure, at Block 725, the exposure of the neural structure may be surgically closed in one embodiment of the present invention. In one embodiment, the insulation form is removed after the insulation cures and before the exposed neural structure is closed. In another embodiment, the insulation form is allowed to remain in the patient's body. Where the insulation form remains in the body, it is preferred that the form is biodegradable in the body. The electrode 200 may be allowed to remain in the patient's body for a pre-determined time period, as indicated in Block 730.

Referring to FIG. 6, a stylized representation for implementing a method of providing a sealed insulation capsule for preventing intercellular body fluids outside the neural structure from contacting the electrode 200 is depicted in accordance with one embodiment of the present invention. To couple an electrode, such as the electrode 200 shown in FIG. 2, to a neural structure, such as a cranial nerve, the lead assembly 235 may be provided at Block 800. For the purposes of covering the electrode 200 and at least a portion of the cranial nerve, at Block 805, liquid insulation may be applied. By allowing the liquid insulation to solidify or thicken, a sealed insulation capsule may be provided, as shown in Block 810.

The sealed insulation capsule may prevent intercellular body fluids outside the neural structure from contacting the electrode 200 in some embodiments of the present invention. The electrode 200 may be used to sense electrical activity on the tissue of the nerve structure, such as the nerve 220 shown in FIG. 3. Alternatively, a therapeutic electrical signal may be applied to the nerve 220 using the insulated electrode 200 for providing a desired therapeutic effect without affecting non-targeted portions of the neurotissue.

The electrode 200 may be implanted within a patient's body to sense a neuropotential associated with the nerve 220. The electrode 200 may be implanted within the patient's body to stimulate the nerve 220. For example, such an implanted electrode may provide neurostimulating to a vagus nerve, in some embodiments of the present invention. By applying an insulating liquid layer proximate the interface between the electrode 200 and a desired portion of the nerve 220, the insulating barrier 230 may be formed.

Alternatively, an insulating sheet may form the insulating barrier 230 for the electrode 200 and a desired portion of the nerve 220 to substantially surround the electrode 200. However, the insulating sheet may be implanted separately from implanting the electrode 200. To implant the insulating sheet, an electrode lead wire assembly, such as a spine may be coupled to the insulating sheet.

By using the insulator 225 to cause the electrode 200 to deliver a stimulation current to a desired portion of a nerve trunk within the patient's body, spread of the stimulation current may be reduced to an undesired portion of the nerve trunk during stimulation of the nerve 220. The insulator 225 may further cause the electrode 200 to reduce electrical contact with the undesired portion of the nerve trunk and/or with intercellular body fluids outside the neural structure within the patient's body.

By using the insulator 225 to cause the electrode 220 to make relatively better (lower impedance) electrical contact to a desired portion of a nerve trunk within the patient's body as compared to the (higher impedance) electrical contact made to surrounding tissues, the electro-potential present on the nerve trunk may be better detected by the implantable device. This improved detection may allow more accurate assessment of the detected information, facilitating improved decision making either by the implantable device itself, or a physician interpreting the detected information.

In one embodiment, sensing and/or stimulation electrodes may be insulated in-situ using a sealing product, such as Kwik-Sil™ marketed by WPI Incorporated of Sarasota, Fla., USA. Such an in-situ sealing agent or material may enable desired recording of endogenous action potentials, and desired containment of stimulation current to a targeted nerve fiber. The insulant may minimize noise and provide a desired seal to enable the IMD 600 to better sense electrical activity on the nerve. In other words, the insulant may provide two primary functions in sensing: one, it insulates the electrodes from extraneous noise in the rest of the body; and two, it prevents the electrical signal (i.e., the differential potential on the nerve body itself) from taking alternate paths from one point to the next. Upon applying a pair of electrodes on the nerve 220, at a certain distance, a voltage differential appears between the two electrodes because of the ion flow in and out of the nerve 220. This current that flows between such two points is divided between an amplifier inside the IMD 600 device that senses the current while the intracellular fluid also conducts that current. So a limited number of electrons may flow from a low potential and a high potential, and if a certain portion of the electrons go off into the intracellular fluid and not go through the amplifier, the amplification of an available signal becomes substantially difficult. Although the same voltage is present, because some of the current is being attenuated at such a low levels, the current flow from intercellular fluids becomes crucial to the functioning of the amplifier. By insulating the nerve from these fluids and preventing that alternate route for the current to flow, much more of the current flows through the amplifier, thereby causing the amplifier to function relatively better.

In stimulating applications, however, the insulant may provide blocking of side effects when an extraneous current may flow through the intracellular fluid into other tissues. In this way, the insulant has two distinct advantages, one in terms of stimulation and focusing the stimulation and the other one is in terms of enabling the sensing of signals on the nerve structure.

In one embodiment, an insulant may be simply “applied” to the back side, i.e., the side which faces away from the nerve structure as opposed to the front side that is in contact with the nerve structure of appropriate electrodes, and form a capsule according to the shape of the surrounding tissue and voids. In other words, the contacting surface of the electrode may be referred to as the front side and then the back side may refer to the exteriorly facing side or exteriorly directed surface.

In another embodiment, long term implantation may be performed by containing the insulant in a controlled shape and size: for durability of the implant, comfort of the patient, and/or repeatability of results from one procedure to the next. A long-term therapeutic period may be a time period ranging from few months to years.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. The particular embodiments disclosed above may be altered or modified and all such variations are considered within the scope and spirit of the invention. Accordingly, the protection sought herein is as set forth in the claims below.

What is claimed:

1. A method for insulating an electrode implanted in a body of a patient, the method comprising:
surgically exposing a nerve of the patient;
implanting at least one electrode in the body of the patient, wherein said implanting comprises coupling said at least one electrode to said nerve of the patient;
providing a mold form;
disposing said mold form around at least a portion of said at least one electrode and said nerve;
introducing into said mold form a curable liquid insulant;
allowing said curable liquid insulant to cure;
removing said mold form from around said at least one electrode and from said patient's body; and
surgically closing said exposure of said nerve.

2. The method of claim 1, wherein implanting at least one electrode comprises implanting a lead having a proximal and a distal end, and wherein said distal end of said lead comprises at least one electrode, said method further comprising:
providing an implantable medical device;
implanting said implantable medical device in the body of the patient; and
coupling said proximal end of said lead to said implantable medical device.

3. The method of claim 1 wherein exposing said nerve comprises exposing a cranial nerve selected from the group consisting of a vagus nerve, a trigeminal nerve, a glossopharyngeal nerve, and branches of the foregoing.

4. The method of claim 1, wherein introducing said curable liquid insulant comprises introducing at least one of a polymer mixture and a gel into the insulation form.

5. The method of claim 1, wherein allowing said curable liquid insulant to cure comprises at least one of cross-linking said curable liquid insulant, thickening said curable liquid insulating, and evaporating a solvent from said curable liquid insulant.

6. The method of claim 1 wherein said mold form comprises a material selected from a rigid sheath, a semi-rigid sheath, and a flexible sheath.

7. An implanted medical device system for treating a medical condition of a patient, the implanted medical device system comprising:
a medical device implanted in the body of the patient for at least one of generating a therapeutic electrical signal and sensing electrical activity on a nerve;
an implanted electrode coupled to a nerve and to the implanted medical device; and
an in situ formed sealed insulation capsule covering the implanted electrode and at least a portion of the nerve to prevent intercellular body fluids outside the nerve from contacting the implanted electrode.

8. The implanted medical device system of claim 7, wherein said implanted electrode is a circumneural electrode such that said insulator substantially surrounds said at least a portion of said nerve.

9. The implanted medical device system of claim 8, wherein said implanted electrode further comprises:
a first helical electrode that surrounds a first portion of said nerve to form a positive electrode; and
a second helical electrode that surrounds a second portion of said nerve to form a negative electrode.

10. The implanted medical device system of claim 7, wherein said medical device further comprises:
a controller to selectively perform an operation selected from the group consisting of providing a therapeutic electrical signal to said implanted electrode and sensing electrical activity on the nerve.

11. The implanted medical device system of claim 7, further comprising a lead coupled to said medical device and to said implanted electrode, wherein said implanted electrode further comprises a plurality of insulated electrodes, each insulated electrode corresponding to a stimulation or sensing channel.

12. The implanted medical device system of claim 7, wherein said sealed insulation capsule is capable of providing strain relief between said proximal end of said lead and said implanted electrode.

13. A method of insulating an implanted electrode coupled to a nerve, the method comprising:
providing a lead assembly comprising at least one lead body having proximal and distal ends, and at least one electrode coupled to said distal end;
surgically exposing a cranial nerve; implanting said lead assembly in the body of the patient, wherein implanting comprises coupling the at least one electrode to the cranial nerve;
providing a mold form disposed around said at least one electrode and around at least a portion of said cranial nerve;
introducing into said mold form a liquid insulation comprising at least one of a polymer mixture and a gel;
curing said liquid insulation to form a sealed insulation capsule around said at least one electrode; and
surgically closing said exposure of said cranial nerve.

14. The method of claim 13, wherein curing said liquid insulation comprises at least one of cross-linking said liquid insulation, thickening said liquid insulation, and evaporating a solvent from said liquid insulation to provide a sealed insulation capsule preventing intercellular body fluids outside said cranial nerve from contacting the at least one electrode.

15. The method of claim 13 further comprising allowing the at least one electrode to remain in the patient's body for a predetermined time period.

16. The method of claim 15, further comprising allowing the at least one electrode to remain in the patient's body for at least one month.

17. The method of claim 13 further comprising removing said mold form from the patient's body.

18. The method of claim 13, further comprising providing a medical device;
implanting said medical device in the body of the patient; and
coupling said medical device to said proximal end of said lead assembly.

19. The method of claim 18, further comprising performing an operation selected from the group consisting of generating a therapeutic electrical signal using said medical device and applying said signal to said at least one electrode, and sensing electrical activity on said cranial nerve.

20. The method of claim 13, wherein said cranial nerve is selected from the group consisting of a vagus nerve, a trigeminal nerve, a glossopharyngeal nerve, and branches of the foregoing.
